(19) United States
(12) Patent Application Publication
(10) Pub. No.: US 2005/0238589 A1
(43) Pub. Date: Oct. 27, 2005

(54) Methods and compositions for preventing or treating periodontal diseases

(76) Inventors: Thomas E. Van Dyke, West Roxbury, MA (US); Nicos A. Petasis, Hacienda Heights, CA (US); Charles N. Serhan, Needham, MA (US)

Correspondence Address: CHOATE, HALL & STEWART LLP, TWO INTERNATIONAL PLACE, BOSTON, MA, 02110, US

(21) Appl. No.: 11/106,141

(22) Filed: Apr. 14, 2005

Related U.S. Application Data
(60) Provisional application No. 60/562,099, filed on Apr. 14, 2004.

(51) Int. Cl. A61K 7/16; A61K 31/185; A61K 31/17; A61K 31/16

(52) U.S. Cl. 424/49; 514/560; 514/553; 514/588; 514/600; 514/627

(57) ABSTRACT

Methods and compositions for preventing or treating periodontal diseases including gingivitis and periodontitis are provided. The invention also provides methods for preventing or treating secondary diseases within or beyond the oral cavity that are related to periodontal disease.

### PRIORITY INFORMATION

The present application claims the benefit of U.S. Ser. No. 60/562,099 filed Apr. 14, 2004, the entire contents of which are hereby incorporated by reference.

### GOVERNMENT FUNDING

This invention was made with Government support under Contract Nos. DE 13499 and GM38765 awarded by the National Institutes of Health. The government has certain rights in this invention.

### BACKGROUND OF THE INVENTION

Periodontal diseases, ranging from gingivitis to more severe forms of periodontitis, are initiated by a bacterial infection followed by a host response that may lead to a highly degenerative oral disease including tooth loss and tissue damage (Page, R. C. (1998) Ann. Periodontol. 3, 108).

Although bacteria appear to be essential for the causation of periodontitis, progression of periodontal disease is dependent on the host response to pathogens that colonize the tooth surface (Hart, T. C., et al. (1994) J. Periodontol. 65, 521). In turn, periodontal disease can be controlled chemotherapeutically by uncoupling host-mediated destruction rather than reducing the etiological load (Offenbacher, S. et al. (1993) J. Periodontol. 64, 432). Along these lines, a body of evidence has identified the inhibition of prostaglandin E₂ (PGE₂) formation and its presence at gingival sites as being relevant therapeutic interventions. For example, PGE₂ generation from gingival homogenates is significantly inhibited by the NSAID flurbiprofen (ElAttar, T. M. A., et al. (1984) J. Periodontol. 55, 536), and cyclooxygenase-derived (COX-derived) eicosanoids in crevicular fluid (CF) are decreased in animals taking flurbiprofen (Smith, M. A., et al. (1993) Infection and Immunity 61, 1453; Offenbacher, S., et al. (1989) J. Periodontal Res. 24, 63). Flurbiprofen also reduced CF-PGE₂ levels, gingival inflammation, tooth attachment loss and bone loss, and in some cases resulted in bone gain (Pauletto, N. et al. (1997) J. Can. Dent. Assoc. 63, 824). In humans, flurbiprofen dramatically decreased the CF-PGE₂ levels (Abramson, M. M. et al. (1992) J. Periodont. Res. 27, 539). These findings suggest that NSAIDs such as flurbiprofen may exert their pharmacological action of inhibiting COX-derived proinflammatory eicosanoids within the periodontium.

Polymorphonuclear leukocytes (PMN, neutrophils) are the most abundant immune cells recruited to early inflammatory periodontal lesions and are the most numerous host cells within the periodontal tissues (Hart, T. C., et al. (1994) J. Periodontol. 65, 521). PMN participate in host defense against bacterial infections and are also involved in noxious inflammatory reactions (Weiss, S. J., et al. (1981) J. Clin. Invest. 68, 714; Babior, B. M. (1984) Blood 64, 959). Recruitment of neutrophils to the periodontium contributes to the progression of periodontal disease and to the destruction of periodontal tissues (Page, R. C. (1998) Ann. Periodontol. 3, 108; Daniel, M. A., et al. (1996) J. Periodontol. 67, 1070). This host response can also be further amplified by the release of an array of inflammatory mediators by neutrophils within the periodontium.

Several inflammatory mediators such as cytokines, chemokines and metalloproteases are associated with periodontal disease (Romanelli, R., et al. (1999) Infect. Immun. 67, 2319; Gainet, J., et al. (1998) Lab. Invest. 78, 755; Assuma, R., et al. (1998) J. Immunol. 160, 403). Other prominent mediators are the arachidonic acid derived products, including leukotriene B₄ (LTB₄) and prostaglandin E₂ (PGE₂) (Offenbacher, S. et al. (1986) J. Periodontal Res. 21, 101). Indeed, many of the pathophysiological events that occur in periodontal diseases can be explained to a large extent by the activities of lipid mediators (Solomon, L. M., et al. (1968) J. Invest. Dermatol. 51, 280; Raisz, L. G., et al. (1974) Prostaglandins 8, 377; Klein, D. C., et al. (1970) Endocrinology 86, 1436; Crunkhorn, P., et al. (1969) Br. J. Pharmacol. 36, 216; Collier, J. G., et al. (1972) Br. J. Pharmacol. 44, 374). For example, LTB₄, a well appreciated and potent chemoattractant, also initiates the accumulation of leukocytes within inflamed sites, stimulates the release of granule-associated enzymes (Borgeat, P., et al. (1990) Clin. Biochem. 23, 459) and was recently found to stimulate bone resorption (Traianedes, K., et al. (1998) Endocrinology 139, 3178).

Along these lines, PGE₂ is a very potent stimulator of bone loss, which is held to be a hallmark of periodontal disease (Zubery, Y., et al. (1998) Infect. Immun. 66, 4158). PGE₂ is also well appreciated for its ability to directly mediate vasodilation, increase vascular permeability, enhance pain perception by bradykinin and histamine, alter connective tissue metabolism, and enhance osteodastic bone resorption (Tsai, C.-C. et al. (1998) J. Dentistry 26, 97). The levels of PGE₂ are significantly elevated in the crevicular fluid of patients with periodontal infections, especially localized juvenile periodontitis, when compared to healthy sites. These levels correlate with disease severity and aggressiveness and constitute a reliable indicator of ongoing clinical periodontal tissue destruction (Offenbacher, S., et al. (1984) J. Periodontal Res. 19, 1). CF-PGE₂ levels can also be used to predict future acute loss of periodontal attachment (Offenbacher, S., et al. (1986) J. Periodontal Res. 21, 101).

Pathophysiological responses that occur in periodontal diseases, including inflammatory cell recruitment, edema, pain, bone resorption and collagen destruction, can be mediated for the most part by effector molecules originating from the arachidonate cascade (Solomon, L. M. et al. (1968) J. Invest. Dermatol. 51, 280; Raisz, L. G., et al. (1974) Prostaglandins 8, 377; Klein, D. C., et al. (1970) Endocrinology 86, 1436; Crunkhorn, P., et al. (1969) Br. J. Pharmacol. 36, 216; Collier, J. G., et al. (1972) Br. J. Pharmacol. 44, 374). In particular, considerable evidence has demonstrated the importance of PGE₂ in the pathogenesis of periodontal diseases. In vitro, PGE₂ increases osteoclast numbers and bone resorption (Lader, C. S., et al. (1998) Endocrinology 139, 3157), decreases proteoglycan synthesis and increases metalloprotease production by cultured chondrocytes (Debrumfemandes, A. J., et al. (1996) Br. J. Pharmacol. 188, 1597). Bone resorption in vivo caused by three periodontal pathogens is mediated in part by PGE₂, causing tooth attachment loss and bone loss (Zubery, Y., et al. (1998) Infect. Immun. 66, 4158). Prior to these findings, PGE₂ was proposed as a reliable molecular indicator of ongoing periodontal tissue destruction that might be used to predict future acute periodontal attachment loss (Offenbacher, S., et al. (1986) J. Periodontal Res. 21, 101).

Prostaglandin endoperoxide synthase (cyclooxygenase, COX) catalyzes two reactions by which arachidonic acid is converted to PGH₂, the common precursor of all prostanoids including PGE₂. To date, two COX isoforms are known (Smith, W. L., et al. (1996) J. Biol. Chem. 271, 33157). COX-1 appears to support the levels of prostanoid biosynthesis required for maintaining organ and tissue homeostasis (Smith, W. L., et al. (1996) J. Biol. Chem. 271, 33157; Vane, J. R., et al. (1996) Scand. J. Rheumatol. 102, 9), whereas COX-2 expression appears to be restricted in basal conditions within most tissues and is up-regulated during inflammation or stress in a wide range of tissues (O'Banion, M. K., et al. (1992) Proc. Natl. Acad. Sci. USA 89, 4888; Seibert, K., et al. (1994) Proc. Natl. Acad. Sci. USA 91, 12013; Needleman, P., et al. (1997) J. Rheumatol. 24, 6). The finding that homogenates of inflamed periodontal tissues display an increased PGE₂ synthetic capacity when compared to homogenates from healthy tissues suggests an increased COX-2 activity is associated with periodontal tissues (ElAttar, T. M. A. (1976) Prostaglandins 11, 331; Albers, H. K., et al. (1979) Dtsch. Zahnarztl. Z. 34, 440; ElAttar, T. M. A., et al. (1982) Prostaglandins Leukot. Med. 8, 447; ElAttar, T. M. A., et al. (1984) J. Periodontol. 55, 536).

The current treatments of periodontal diseases involve primarily the use of compositions containing antimicrobial compounds or various non-steroidal antiinflammatory agents (NSAIDs). Despite these known treatments, there remains a need for novel methods and compositions for treating and preventing periodontal diseases.

### SUMMARY OF THE INVENTION

Methods and compositions for preventing or treating periodontal diseases including gingivitis and periodontitis are provided.

The inventive methods comprise administering to a subject a prophylactically or therapeutically effective amount of a compound of formula I: 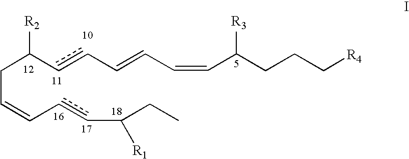
or a pharmaceutically acceptable salt or prodrug thereof, wherein:
each 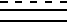
   independently designates a double or triple bond;
R¹, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or:
   two R on the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O)R, NRC(O)N(R)₂, C(O)OR, C(O)N(R)₂, SO₂R, NRSO₂R, C(O)R, or SO₂N(R)₂.

In certain embodiments, the compound of formula I is administered in a pharmaceutical composition with a pharmaceutically acceptable carrier. These pharmaceutical compositions may optionally comprise one or more additional therapeutic agents. In certain embodiments, the additional therapeutic agent or agents are antimicrobial compounds and/or non-steroidal antiinflammatory agents (NSAIDs). In certain preferred embodiments, the additional therapeutic agent or agents are COX-2 inhibitors, preferably selective COX-2 inhibitors, e.g., celecoxib, rofecoxib, and/or valdecoxib. The invention also provides methods for preventing or treating secondary diseases within or beyond the oral cavity that are related to periodontal disease. The invention further provides pharmaceutical compositions for preventing or treating periodontal diseases including gingivitis and periodontitis. These pharmaceutical compositions include a compound of formula I or a pharmaceutically acceptable salt or prodrug thereof and a pharmaceutically acceptable carrier.

### DESCRIPTION OF THE DRAWING

FIG. 1 shows photographs of the buccal and lingual mandibles of rabbits treated either by ligature alone (A), ligature with topical Porphyromonas gingivalis (P. gingivalis) application and vehicle (B), or ligature with topical P. gingivalis application and resolvin E1 (C). Arrows indicate the levels of gingiva (A1-C1 and A3-C3) and bone (A2-C2) and (A4-C4).

FIG. 2 shows graphs of alveolar bone levels of defleshed bone specimens after the different treatments of FIG. 1. Both proximal and crestal sites were measured for the buccal and lingual sides.

FIG. 3 show radiographs of bone and other hard tissue components of rabbits treated by ligature alone (A), ligature with topical P. gingivalis application and vehicle (B), or ligature with topical P. gingivalis application and resolvin E1 (C). Arrows indicate sites of bone loss.

FIG. 4 shows a graph of the percentage of tooth in bone as measured on the radiographs of FIG. 3.

FIG. 5 shows a graph of the percentage of bone loss as calculated from the radiographs of FIG. 3 by the Bjorn technique.

FIG. 6 shows histological images of samples taken from rabbits treated either by ligature alone (A), ligature with topical P. gingivalis application and vehicle (B), or ligature with topical P. gingivalis application and resolvin E1 (C).

### Definitions

As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, compounds of the invention may optionally be substituted with one or more substituents. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or branched, hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”, “carbocycle” or “cycloalkyl”), and has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃₋₈ hydrocarbon or bicyclic C₈₋₁₂ hydrocarbon that is completely saturated or that contains one or more units of unsaturation, but which is not aromatic, and has a single point of attachment to the rest of the molecule wherein any individual ring in said bicyclic ring system has 3-7 ring members. Suitable aliphatic groups include, but are not limited to, linear or branched, alkyl, alkenyl, alkynyl groups and hybrids thereof such as (cycloalkyl)alkyl, (cycloalkenyl)alkyl or (cycloalkyl)alkenyl. In other embodiments, an aliphatic group may have two geminal hydrogen atoms replaced with oxo (a bivalent carbonyl oxygen atom ═O), or a ring-forming substituent, such as —O-(straight or branched alkylene or alkylidene)-O— to form an acetal or ketal.

In certain embodiments, exemplary aliphatic groups include, but are not limited to, ethynyl, 2-propynyl, 1-propenyl, 2-butenyl, 1,3-butadienyl, 2-pentenyl, vinyl (ethenyl), allyl, isopropenyl, methyl, ethyl, propyl, isopropyl, butyl, isobutyl, sec-butyl, tert-butyl, pentyl, isopentyl, sec-pentyl, neo-pentyl, tert-pentyl, cyclopentyl, hexyl, isohexyl, sec-hexyl, cyclohexyl, 2-methylpentyl, tert-hexyl, 2,3-dimethylbutyl, 3,3-dimethylbutyl, 1,3-dimethylbutyl, and 2,3-dimethyl but-2-yl.

The term “haloaliphatic” means aliphatic substituted with one or more halogen atoms. The term “halogen” means F, Cl, Br, or I. Such “haloaliphatic” groups may have two or more halo substituents which may or may not be the same halogen and may or may not be on the same carbon atom. Examples include chloromethyl, periodomethyl, 3,3-dichloropropyl, 1,3-difluorobutyl, trifluoromethyl, and 1-bromo-2-chloropropyl.

The term “heterocycle”, “heterocyclyl”, “heterocycloaliphatic”, or “heterocyclic” as used herein means non-aromatic, monocyclic, bicyclic, or tricyclic ring systems in which one or more ring members is an independently selected heteroatom. In some embodiments, the “heterocycle”, “heterocyclyl”, “heterocycloaliphatic”, or “heterocyclic” group has 3-14 ring members in which one or more ring members is a heteroatom independently selected from oxygen, sulfur, nitrogen, or phosphorus, and each ring in the system contains 3-7 ring members.

The term “heteroatom” means one or more of oxygen, sulfur, nitrogen, phosphorus, or silicon (including, any oxidized form of nitrogen, sulfur, phosphorus, or silicon); the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units of unsaturation.

The term “aryl” used alone or as part of a larger moiety as in “aralkyl”, “aralkoxy”, or “aryloxyalkyl”, refers to monocyclic, bicyclic, and tricyclic ring systems having a total of 5-14 ring members, wherein at least one ring in the system is aromatic and wherein each ring in the system contains 3-7 ring members. The term “aryl” may be used interchangeably with the term “aryl ring”. The term “aryl” also refers to heteroaryl ring systems as defined hereinbelow.

The term “heteroaryl”, used alone or as part of a larger moiety as in “heteroaralkyl” or “heteroarylalkoxy”, refers to monocyclic, bicyclic, and tricyclic ring systems having a total of 5-14 ring members, wherein at least one ring in the system is aromatic, at least one ring in the system contains one or more heteroatoms, and wherein each ring in the system contains 3-7 ring members. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from halogen; N₃, CN, R^{o}; OR^{o}; SR^{o}; 1,2-methylene-dioxy; 1,2-ethylenedioxy; phenyl (Ph) optionally substituted with R^{o}; —O(Ph) optionally substituted with R^{o}; (CH₂)₁₋₂(Ph), optionally substituted with R^{o}; CH═CH(Ph), optionally substituted with R^{o}; NO₂; CN; N(R^{o})₂; NR^{o}C(O)R^{o}; NR^{o}C(O)N(R^{o})₂; NR^{o}CO₂R^{o}; —NR^{o}NR^{o}C(O)R^{o}; NR^{o}NR^{o}C(O)N(R^{o})₂; NR^{o}NR^{o}CO₂R^{o}; C(O)C(O)R^{o}; C(O)CH₂C(O)R^{o}; CO₂R^{o}; C(O)R^{o}; C(O)N(R^{o})₂; OC(O)N(R^{o})₂; S(O)₂R^{o}; SO₂N(R^{o})₂; S(O)R^{o}; NR^{o}SO₂N(R^{o})₂; NR^{o}SO₂R^{o}; C(═S)N(R^{o})₂; C(═NH)—N(R^{o})₂; or (CH₂)₀₋₂NHC(O)R^{o} wherein each independent occurrence of R^{o} is selected from hydrogen, optionally substituted C₁₋₆ aliphatic, an unsubstituted 5-6 membered heteroaryl or heterocyclic ring, phenyl, O(Ph), or CH₂(Ph), or, notwithstanding the definition above, two independent occurrences of R^{o}, on the same substituent or different substituents, taken together with the atom(s) to which each R^{o} group is bound, form a 3-8 membered cycloalkyl, heterocyclyl, aryl, or heteroaryl ring having 0-4 heteroatoms independently selected from nitrogen, oxygen, or sulfur. Optional substituents on the aliphatic group of R^{o} are selected from N₃, CN, NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo C₁₋₄ aliphatic, wherein each of the foregoing C₁₋₄ aliphatic groups of R^{o} is unsubstituted.

An aliphatic or heteroaliphatic group or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

Optional substituents on the nitrogen of a non-aromatic heterocyclic ring are selected from R⁺, N(R⁺)₂, C(O)R⁺, CO₂R⁺, C(O)C(O)R⁺, C(O)CH₂C(O)R⁺, SO₂R⁺, SO₂N(R⁺)₂, C(═S)N(R⁺)₂, C(═NH)—N(R⁺)₂, or NR⁺SO₂R⁺; wherein R⁺ is hydrogen, an optionally substituted C₁₋₆ aliphatic, optionally substituted phenyl, optionally substituted O(Ph), optionally substituted CH₂(Ph), optionally substituted (CH₂)₁₋₂(Ph); optionally substituted CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R⁺, on the same substituent or different substituents, taken together with the atom(s) to which each R⁺ group is bound, form a 3-8-membered cycloalkyl, heterocyclyl, aryl, or heteroaryl ring having 0-4 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄ aliphatic groups of R⁺ is unsubstituted.

As detailed above, in some embodiments, two independent occurrences of R^{o} (or R⁺, or any other variable similarly defined herein), are taken together with the atom(s) to which each variable is bound to form a 3-8 membered cycloalkyl, heterocyclyl, aryl, or heteroaryl ring having 0-4 heteroatoms independently selected from nitrogen, oxygen, or sulfur. Exemplary rings that are formed when two independent occurrences of R^{o} (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of OR^{o} 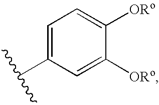
these two occurrences of R^{o} are taken together with the oxygen atoms to which they are bound to form a fused 6-membered oxygen containing ring: 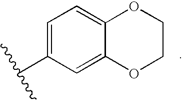
It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As used herein, “pharmaceutically acceptable salts or prodrugs” are salts or prodrugs that are, within the scope of sound medical judgment, suitable for use in contact with the tissues of patients without undue toxicity, irritation, allergic response, and the like, commensurate with a reasonable benefit/risk ratio, and effective for their intended use. These compounds include the zwitterionic forms, where possible, of compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like (see, e.g., Berge S. M., et al. (1977) J. Pharm. Sci. 66, 1, which is incorporated herein by reference).

The term “prodrug” refers to compounds that are rapidly transformed in vivo to yield a compound of formula I, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of a compound, to mask side effects or toxicity, to improve the flavor of a compound or to alter other characteristics or properties of a compound. By virtue of knowledge of pharmacodynamic processes and drug metabolism in vivo, once a pharmaceutically active compound is identified, those of skill in the pharmaceutical art generally can design prodrugs of the compound (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

### Description of Certain Preferred Embodiments

Methods for preventing or treating periodontal diseases including gingivitis and periodontitis are provided. The methods comprise administering to a subject a prophylactically or therapeutically effective amount of a compound of formula I: 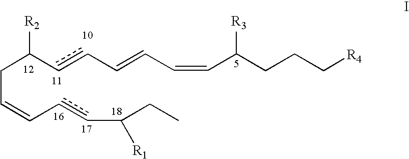
or a pharmaceutically acceptable salt or prodrug thereof, wherein:
each 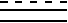
   independently designates a double or triple bond;
R¹, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or:
   two R on the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O)R, NRC(O)N(R)₂, C(O)OR, C(O)N(R)₂, SO₂R, NRSO₂R, C(O)R, or SO₂N(R)₂.

As defined generally above, each 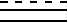
independently designates a double or triple bond. In certain embodiments, the 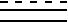
between the C10 and C11 carbons designates a triple bond. In other embodiments, the 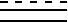
between the C16 and C17 carbons designates a triple bond. In yet other embodiments, the 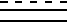
between the C10 and C11 carbons and the C16 and C17 carbons designates a triple bond. In one embodiment each 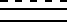
designates a double bond.

As defined generally above, R¹, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂. In certain embodiments, R¹, R², and R³ are each independently OR, OX¹, SR, SX², N(R)₂, or NHX³. In other embodiments, R¹, R², and R³ are each independently OR or OX¹.

As defined generally above, R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or two R on the same nitrogen are taken together with the nitrogen to form a 5-8 membered heterocyclyl or heteroaryl ring having 1-3 heteroatoms independently selected from nitrogen, oxygen, or sulfur. In certain embodiments, R is independently selected from hydrogen or an optionally substituted C₁₋₆ aliphatic group. In one embodiment R is hydrogen.

As defined generally above, R⁴ is NRC(O)R, NRC(O)N(R)₂, C(O)OR, C(O)N(R)₂, SO₂R, NRSO₂R, C(O)R, or SO₂N(R)₂. In certain embodiments, R⁴ is C(O)OR, C(O)N(R)₂ or SO₂R. In one embodiment R⁴ is C(O)OR.

The compound of formula I includes chiral centers at carbon positions 5, 12 and 18. It should be understood that the present invention encompasses the use of all stereochemical forms of this compound as defined above. Therefore, single stereochemical isomers as well as enantiomeric and diastereoisomeric mixtures of the present compounds are within the scope of the invention. In certain embodiments, the C18 carbon has an R configuration. In some of these embodiments, the C5 carbon has an S configuration, the C12 carbon has an R configuration and the C18 carbon has an R configuration. In one such embodiment, each 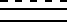
designates a double bond; R¹, R², and R³ are each OH; R⁴ is C(O)OH and the compound of formula I is termed resolvin E1.

Resolvin E1 belongs to an array of natural bioactive lipids that are generated in vivo from ω-3 polyunsaturated fatty acids by aspirin modified COX-2 (Serhan, C. N., et al. (2000) J. Exp. Med. 192, 1197; Serhan, C. N., et al. (2002) J. Exp. Med. 196, 1025, the contents of which are incorporated herein by reference). The use of resolvin E1 is further described in the Examples. In certain embodiments, the compounds of the invention are prepared in vivo or in vitro and then substantially purified and isolated by techniques known in the art (see, for example, U.S. Pat. No. 6,670,396, the contents of which are incorporated herein by reference). Without limitation, the purity of the compounds is generally at least about 90%, preferably at least about 95%, and most preferably at least about 99%. Certain compounds of the invention may also be prepared by chemically modifying one or more purified compounds. For example, a purified compound may be chemically modified into a pharmaceutically acceptable salt or prodrug as described above. Additionally or alternatively, one or more hydroxyl, thiol or amino groups may be protected as further described below. Additionally, in other embodiments, the compounds of formula I are manufactured independently using conventional synthetic methods for preparing lipid derivatives.

Protecting Groups

As described herein, R¹, R², and R³ in the compounds of the invention may include hydroxyl (X¹), thiol (X²) and/or amino (X³) protecting groups. Suitable hydroxyl, thiol and amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference.

Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl. Preferred hydroxyl protecting groups include methyl and ethyl ethers, TMS or TIPPS groups, acetate or proprionate groups and glycol ethers, such as ethylene glycol and propylene glycol derivatives.

Examples of suitable thiol protecting groups include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, trichloroethoxycarbonyl, to name but a few. A preferred thiol protecting group is —S—S-pyridin-2-yl.

Examples of suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group is phthalimido. In other embodiments, the amino protecting group is mono- or di-benzyl or mono- or di-allyl. In still other embodiments, the amino protecting group is a tert-butyloxycarbonyl (BOC) group.

Pharmaceutical Compositions

In certain embodiments, the compound of formula I is administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are antimicrobial compounds and/or non-steroidal antiinflammatory agents (NSAIDs). In certain preferred embodiments, the additional therapeutic agent or agents are COX-2 inhibitors, preferably selective COX-2 inhibitors, e.g., celecoxib, rofecoxib, and/or valdecoxib.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences ed. Gennaro, Mack Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Prophylactically or Therapeutically Effective Amount

The pharmaceutical compositions of the invention may include a “prophylactically effective amount” or a “therapeutically effective amount” of a compound of formula I. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. A “therapeutically effective amount” refers to an amount effective, at dosages and for periods of time necessary, to achieve the desired therapeutic result, e.g., a diminishment or prevention of the symptoms or disease. A therapeutically effective amount of the compound of formula I may vary according to factors such as the disease state, age, sex, and weight of the subject, and the ability of the compound to elicit a desired response in the subject.

A prophylactically or therapeutically effective amount is also one in which any toxic or detrimental effects of the compound are outweighed by the beneficial effects.

The therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is sufficient to reduce inflammation and bone loss in a subject suffering from a periodontal disease. In preferred embodiments, the therapeutically effective amount is sufficient to eliminate inflammation and bone loss in a subject suffering from a periodontal disease.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred.

Administration of Pharmaceutical Compositions

After formulation with an appropriate pharmaceutically acceptable carrier in a desired dosage, the pharmaceutical compositions of this invention can be administered to a subject. As used herein, the term “subject” refers to any living organism in which an, immune response, e.g., an antiinflammatory response can be elicited. The term includes, but is not limited to, humans, nonhuman primates such as chimpanzees and other apes and monkey species; farm animals such as cattle, sheep, pigs, goats and horses; domestic subjects such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The pharmaceutical compositions of this invention can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes. The preferred method of administration is topical, for example, topical delivery to the teeth and/or oral cavity.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, toothpicks, foods, and dental floss that comprise one or more compounds of formula I. Preferred forms of the compositions are those that can be administered topically to the oral cavity and/or teeth. Without limitation, these include toothpastes, chewing gum, mouth sprays, mouthwashes, toothpicks, and dental floss. In a preferred embodiment the composition is in the form of a toothpaste. In another preferred embodiment the composition is in the form of a mouthwash. Exemplary methods for preparing suitable compositions for topical delivery to the oral cavity and teeth are described in U.S. Pat. Nos. 6,706,256; 6,214,320 and 5,827,503 (the entire contents of which are incorporated herein by reference). Additionally, the compounds of the invention can be conjugated with or covalently linked to polymers, such as those that are conventionally used for the manufacture of dental floss. The compounds can also be incorporated into polymers or biopolymers for the sustained release of the compounds. Further, the compounds can be incorporated into liposomes for sustained release delivery.

Uses of Pharmaceutical Compositions

As discussed above and described in greater detail in the Examples, compounds of formula I may be used to prevent or treat periodontal diseases. Prophylactic measures can be taken to forestall the occurrence, or reoccurrence of periodontal disease. Therapeutic measures can be taken to treat periodontal disease once it has occurred.

As used herein, the terms “periodontal diseases” include all diseases of the periodontal tissues that surround and support the teeth (e.g., see Williams D. M. et al., “Pathology of periodontal disease” Oxford University Press, 1992). These include the gingiva, cementum, periodontal ligament, alveolar process bone, and dental supporting bone. Specifically, periodontal diseases include gingivitis and periodontitis. Gingivitis is a disease in which inflammation is localized within the gingiva and no lesion occurs in the bone and periodontal ligament and there is no attachment loss between the teeth and gingiva. Periodontitis is a disease in which gingival inflammation reaches the periodontal ligament and alveolar bone, the pocket becomes a periodontal pocket, and the attachment level (the position of attachment) is on the root apex side downward from the cementum-enamel junction.

It is to be understood that compounds of formula I may also be used to prevent or treat secondary diseases that are related to a periodontal disease. For example, compounds of formula I may be used to prevent or treat secondary diseases of other oral tissues, e.g., without limitation, aphthous ulcers, herpetic stomatitis, etc. Further, periodontal disease has been shown to have implications beyond the deleterious effects on oral tissues and structural integrity. In particular, periodontal disease represents a potential risk factor for increased morbidity or mortality in pregnancy and for several systemic diseases including cardiovascular disease and diabetes (Page, R. C. (1998) Ann. Periodontol. 3, 108; Garcia, R. I., et al. (1998) Ann. Periodontol. 3, 339). In this context, it has been shown that local infection with the periodontal pathogen P. gingivalis up-regulates the expression of COX-2 in lung associated tissues (U.S. Patent Application No. 2004/00191110 by Van Dyke et al., published Jan. 29, 2004) which is a marker of on-going inflammation (Herschman, H. R. (1998) Trends Cardiovasc. Med. 8, 145). In view of these results, the prevention or treatment of periodontal diseases is likely to have a beneficial impact on the prognosis of a number of systemic diseases. Thus, the present invention is also related to methods for treating systemic diseases that are related to periodontal disease, such as cardiovascular diseases, pregnancy complications, and diabetes. These methods also comprise administering to a subject a prophylactically or therapeutically effective amount of a compound of formula I.

### EXAMPLES

The present invention is further illustrated and supported by the following example. However, this example should in no way be considered to further limit the scope of the invention. To the contrary, one having ordinary skill in the art would readily understand that there are other embodiments, modifications, and equivalents of the present invention without departing from the spirit of the present invention and/or the scope of the appended claims.

### Example 1

A. Materials and Methods:

Animal Model

The study protocol and experimental design was reviewed and approved by the Boston University Medical Center Institutional Animal Care and Use Committee (BUMC IACUC) prior to study initiation (IACUC protocol #: 2003-O₂). In addition, BUMC Institutional Biohazard Committee (IBC) approved the use of P. gingivalis in this animal model to induce periodontal disease (IBC protocol #: A-269). In total, 14 New Zealand White rabbits (male, 3.5-4.0 kg each) were used in these experiments. The animals were distributed as follows: Group A: ligature alone (2 rabbits), Group B: ligature+P. gingivalis+resolvin E1 (6 rabbits), Group C: ligature+P. gingivalis+vehicle (6 rabbits). All animals were purchased from Pine Acre Farms. The weight of the animals was strictly controlled and all animals weighed between 3.5-4.0 kg at the time of the initial experimentation. The animals were kept in individual cages, received water ad libitum, and fed with specialized food (chow) for at least 5 days for acclimatization by experienced and licensed laboratory technicians.

Experimental Periodontitis

Ligature placement was performed under general anesthesia using ketamine (4.0 mg/kg) and xylazine (5 mg/kg) injections. Animals had a 3-0 silk suture placed around the second premolar of both mandibular quadrants. Group A only received ligature while Groups B and C received P. gingivalis in addition to ligature placement. P. gingivalis (strain A7436) was grown as previously described. Briefly, bacteria were cultured on agar plates containing trypticase soy agar supplemented with 0.5% (w/v) yeast extract, 5% defibrinated sheep red blood cells, 5 μg hemin, and 1 μg/ml vitamin K. Plates were incubated for 3 days at 37° C. in jars anaerobically maintained through palladium catalyzed hydrogen/carbon dioxide envelopes (GasPak Plus, BD Microbiology Systems, Sparks, Md., USA). Colonies were randomly selected and anaerobically cultured overnight at 37° C. in Schaedler's broth supplemented with vitamin K and hemin. Bacteria numbers were spectrophotometrically determined at 600 nm and 10⁹ CFU (0.8 OD) were mixed with carboxymethylcellulose to form a thick slurry, which was applied topically to the ligated teeth. The sutures were checked at every appointment, and lost or loose sutures were replaced.

Topical Application of Resolvin E1

Topical applications were performed every-other-day for 6 weeks and under the inhalation anesthesia using isoflurane (4.0 MAC/2.0 MAC). In Group B, ethanol (7 μl), which was used as a carrier vehicle for resolvin E1 and in Group C, resolvin E1 (7 μg/μl) suspended in ethanol was applied before the delivery of P. gingivalis. At the end of the study, animals were euthanized using overdose pentobarbital (euthanasia) injections (120 mg/kg) according to the approved protocol by the IACUC. No adverse events were observed during experimental procedures throughout the study with regard to the animal care and no animals were prematurely lost during the study.

Morphometric Analysis

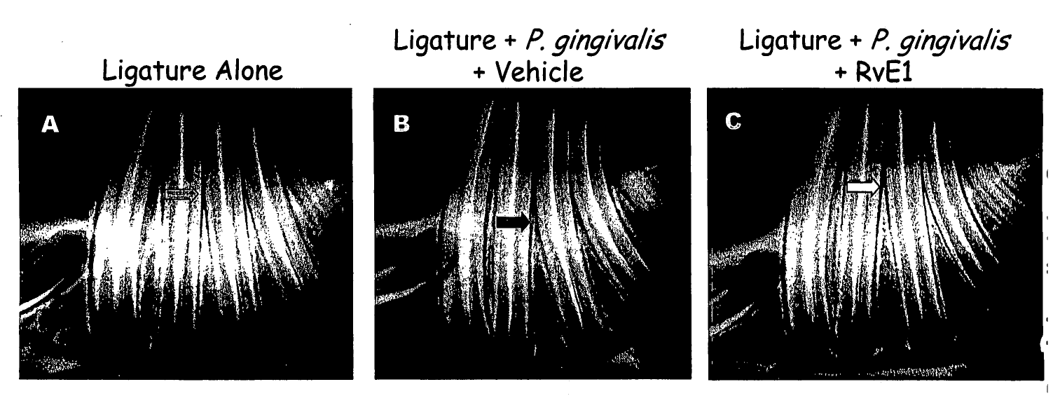

After sacrificing the animals, the mandible was dissected free of the muscles and the soft tissue, keeping the attached gingiva intact with the bone. Then the mandible was split into two halves from the midline between the central incisors. Half was taken for morphometric analysis of the bone loss and the other half was used for histological evaluation of periodontitis. Half of the sectioned mandible was defleshed by immersing in 10% hydrogen peroxide (10 min, room temperature). The soft tissue was removed carefully and then the mandible was stained with methylene blue for good visual distinction between the tooth and the bone (see FIG. 3, A2-C2 and A4-C4). Next, the bone level around the second premolar was measured directly by a 0.5 mm calibrated periodontal probe. Measurements were made at three points each, at buccal and lingual sides, for crestal bone level. A mean crestal bone level around the tooth was calculated. Similarly, for the proximal bone level, measurements were made at mesial and distal aspects of the tooth. The measurements were taken from both the buccal and lingual side on both proximal aspects of the second premolar and the mean proximal bone level was calculated. The bone level was also quantified by Image Analysis (Image-Pro Plus 4.0, Media Cybernetics, Silver Spring, Md.). The sectioned mandible was mounted and photographed using an inverted microscope at 10×. The captured image was also analyzed as above and the mean crestal bone level around the tooth was calculated in millimeters.

Radiographic Analysis

The percentage of the tooth within the bone was calculated radiographically using the Bjorn technique. The radiographs were taken with a digital X-ray (Schick Technologies Inc, Long Island City, N.Y.). To quantify bone loss, the length of the tooth from the cusp tip to the apex of the root was measured, as was the length of the tooth structure outside the bone, measured from the cusp tip to the coronal extent of the proximal bone. From this, the percentage of the tooth within the bone was calculated. Bone values are expressed as the percentage of the tooth in the bone (length of tooth in bone×100/total length of tooth).

Histological Analysis

For histological analysis, the other half of the mandible was immersed in a volume of Immunocal (Decal Corporation, Tallman, N.Y., USA) equal to at least 10 times the size of section; solution was replaced every 24 hours for 72 hours. After the decalcification, the tissues were rinsed for 1-3 minutes in running water, placed in Cal-Arrest (Decal Corporation, Tallman, N.Y., USA) in order to neutralize the pH of the tissue, enhance embedding and staining characteristics, and stop further decalcification so that the tissue does not become over-decalcified. The tissue was kept in this solution for 2-3 minutes, rinsed again in flowing deionized water for at least 3 minutes and embedded in paraffin. Thin sections (0.7 μm) were cut and stained with hematoxylin and eosin (HE) to identify the cellular composition of the inflammatory infiltrates.

B. Results:

Morphometric Analysis

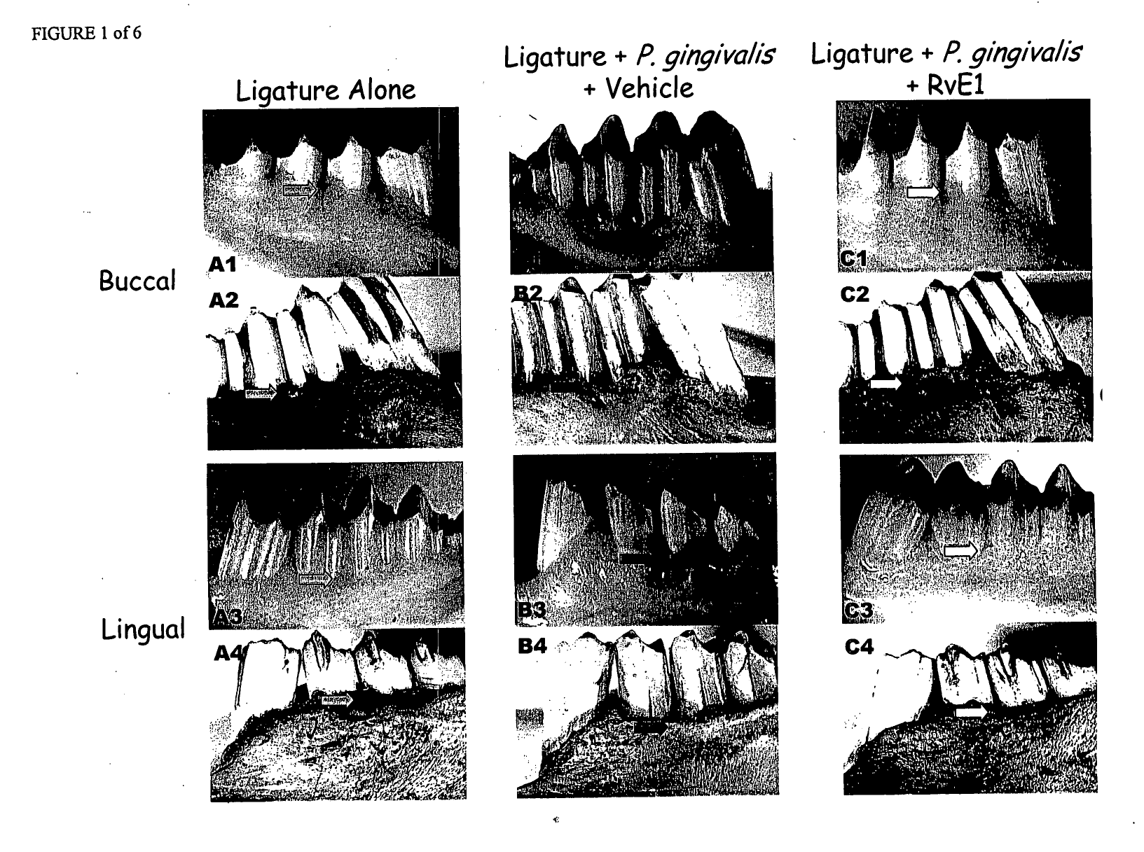

FIG. 1 shows the mandibles of rabbits treated either by ligature alone or ligature and topical P. gingivalis application and then received either resolvin E1 compound or the vehicle (ethanol). The gingival tissue and defleshed bone specimens from buccal or lingual aspects are shown. Ligature placement without additional P. gingivalis application did not lead to any significant soft or hard tissue changes in rabbit mandibles (A1-A4). Green arrows indicate the “normal” levels of gingiva and bone. In animals, which received P. gingivalis in conjunction with ligature placement, there was a significant gingival inflammation and bone resorption (B1-B4). Red arrows depict the soft and hard tissue changes in this group of animals. Topical delivery of resolvin E1 before P. gingivalis application prevented the gingival inflammation and bone destruction (C1-C4, yellow arrows).

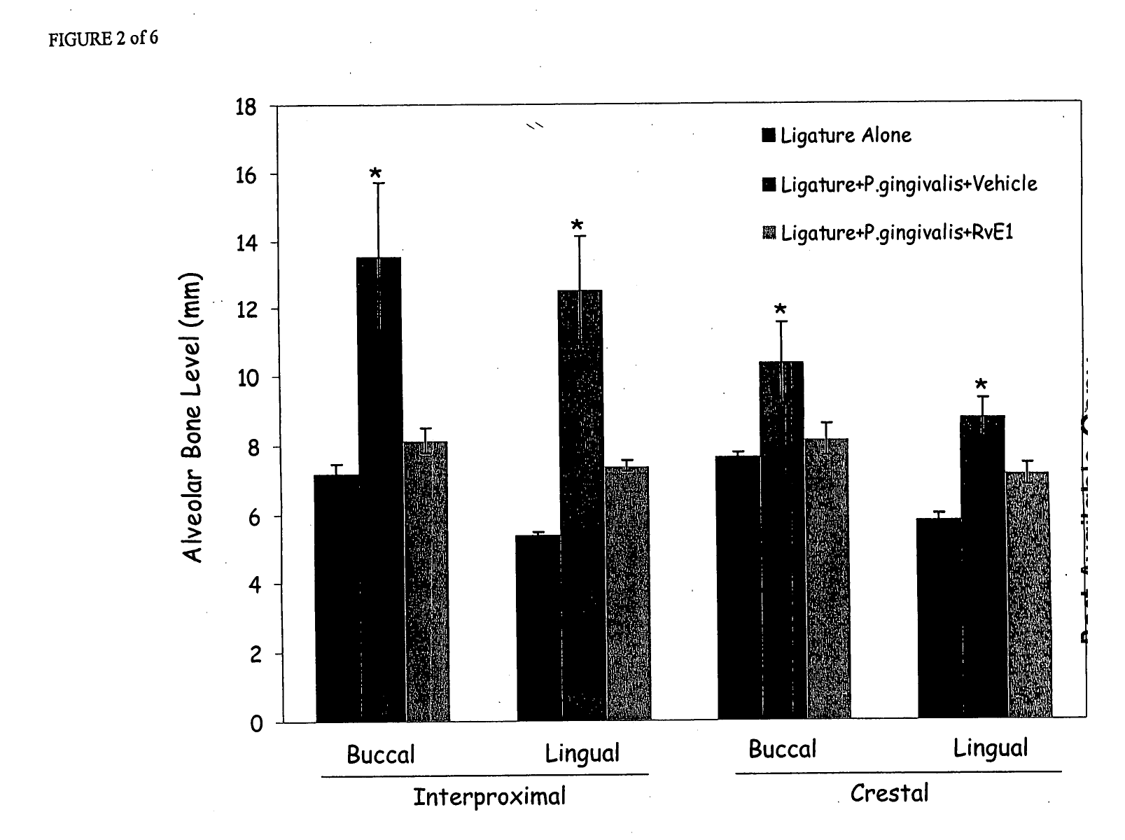

FIG. 2 shows the quantitative analyses of defleshed bone specimens. The findings demonstrate that preventive effects of resolvin E1 on P. gingivalis and ligature-induced experimental periodontitis in rabbits are statistically significant compared to animals that have received the vehicle as placebo where the bone loss was significantly higher (p<0.05, ANOVA).

Radiographic Analysis

FIG. 3 shows the radiographic analyses of bone and other hard tissue components. The bone loss in animals that have received ligature placement, P. gingivalis, and vehicle is shown in (B). The bone loss (red arrow) is visible and significantly different compared to animals that have received ligature alone (A, green arrow). Topical application of resolvin E1 prevented the bone loss and radiographic appearance of alveolar bone was at the same level as those animals that have received the ligature application alone (C, yellow arrows).

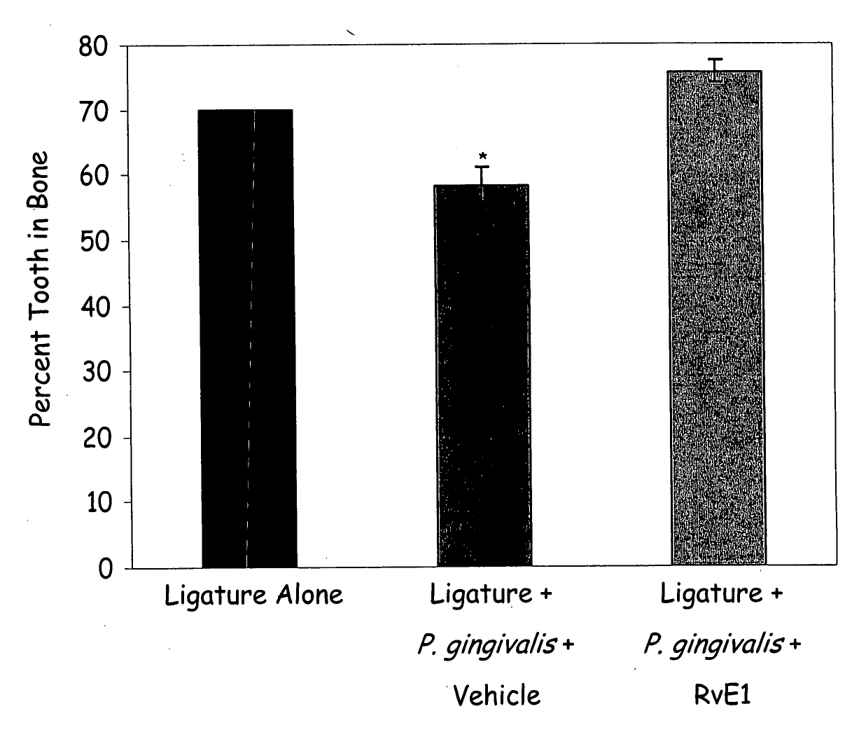

FIG. 4 shows the percentage of tooth in bone as measured on the radiographs of FIG. 3. Resolvin E1 led to maintenance of bone levels in the presence of periodontal disease challenge. P. gingivalis application in conjunction with ligature placement resulted in significant bone loss (* p<0.05 compared to resolvin E1 or ligature alone).

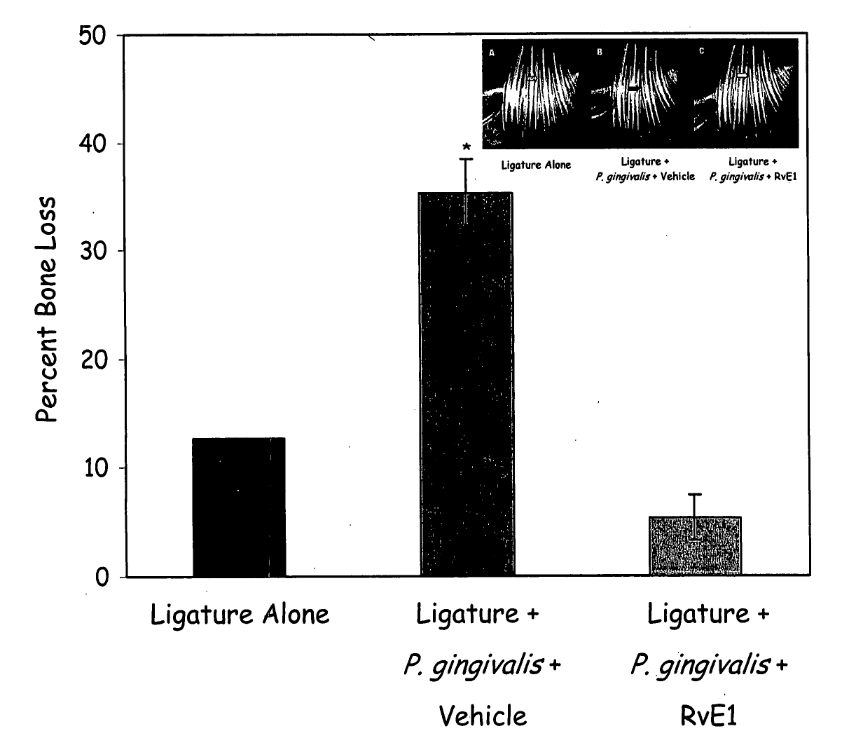

FIG. 5 depicts the percentage of bone loss as calculated by the Bjorn technique. This measurement further confirmed that resolvin E1 application prevents the destructive effects of P. gingivalis-induced periodontitis (* p<0.05 compared to resolvin E1 or ligature alone).

Histological Analysis:

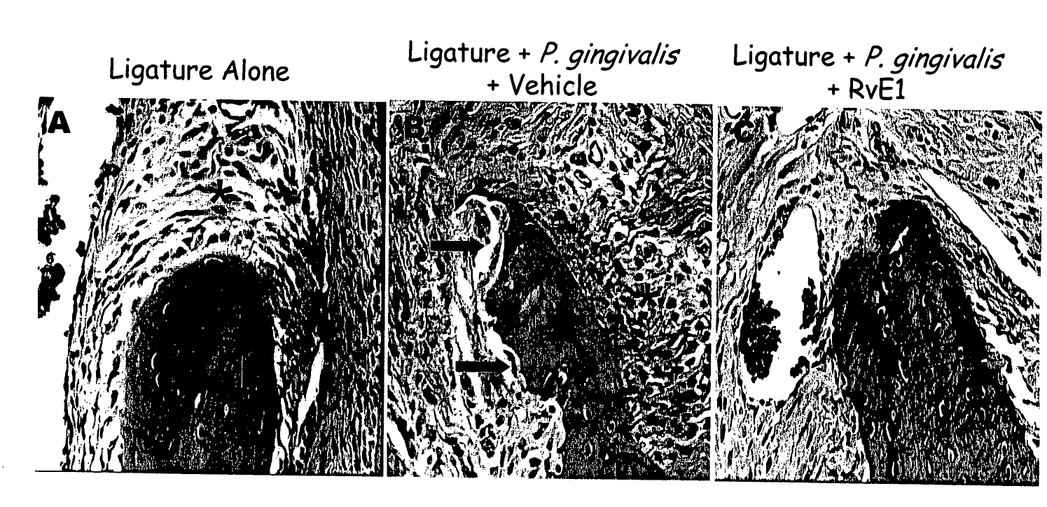

FIG. 6 shows the histological changes in response to different treatments. Ligature placement around the second premolars of rabbit mandible led to increased numbers of inflammatory cells (indicated with an *) while no bone loss nor any osteoclastic activity were visible (A). Local P. gingivalis administration in addition to the ligature placement led to significant bone resorption as depicted by black arrows and increased inflammation (B). Topical application of resolvin E1 prevented both the bone loss and inflammatory changes in rabbits that receive P. gingivalis and ligature placement (C).

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A method for preventing or treating a periodontal disease in a subject comprising administering to a subject a prophylactically or therapeutically effective amount of a compound of formula I: 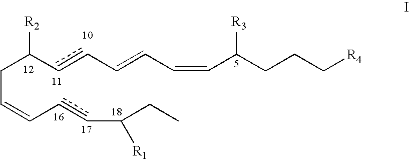
or a pharmaceutically acceptable salt or prodrug thereof, wherein:
each 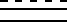
independently designates a double or triple bond;
R¹, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or:
two R on the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O)R, NRC(O)N(R)₂, C(O)OR, C(O)N(R)₂, SO₂R, NRSO₂R, C(O)R, or SO₂N(R)₂.

2. The method of claim 1 wherein the 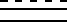
between the C10 and C11 carbons designates a triple bond.

3. The method of claim 1 wherein the 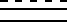
between the C16 and C17 carbons designates a triple bond

4. The method of claim 1 wherein the 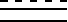
between the C10 and C11 carbons and the C16 and C17 carbons designates a triple bond.

5. The method of claim 1 wherein each 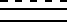
designates a double bond.

6. The method of claim 1 wherein R¹, R², and R³ are each independently OR, OX¹, SR, SX², N(R)₂, or NHX³.

7. The method of claim 6 wherein R¹, R², and R³ are each independently OR or OX¹.

8. The method of claim 1 wherein R is independently selected from hydrogen or an optionally substituted C₁₋₆ aliphatic group.

9. The method of claim 1 wherein R is hydrogen.

10. The method of claim 1 wherein R⁴ is C(O)OR, C(O)N(R)₂, or SO₂R.

11. The method of claim 10 wherein R⁴ is C(O)OR.

12. The method of claim 1 wherein the C18 carbon has an R configuration.

13. The method of claim 1 wherein the C5 carbon has an S configuration, the C12 carbon has an R configuration and the C18 carbon has an R configuration.

14. The method of claim 13 wherein each 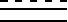
designates a double bond; R¹, R², and R³ are each OH; and R⁴ is C(O)OH.

15. The method of claim 1 wherein the compound of formula I is present within a pharmaceutical composition that includes a pharmaceutically acceptable carrier.

16. The method of claim 15 wherein the pharmaceutical composition further includes an antimicrobial compound or a non-steroidal antiinflammatory compound.

17. The method of claim 15 wherein the pharmaceutical composition further includes a COX-2 inhibitor.

18. The method of claim 17 wherein the COX-2 inhibitor is selected from the group consisting of celecoxib, rofecoxib, and valdecoxib.

19. The method of claim 15 wherein the pharmaceutical composition is administered topically to the subject's oral cavity.

20. The method of claim 19 wherein the pharmaceutical composition is in a form selected from the group consisting of solutions, suspensions, dispersions, ointments, creams, pastes, gels, powders, toothpastes, lozenges, salve, chewing gum, mouth sprays, pastilles, sachets, mouthwashes, aerosols, tablets, capsules, transdermal patches, toothpicks, foods, and dental floss.

21. The method of claim 19 wherein the pharmaceutical composition is in a form selected from the group consisting of toothpastes, chewing gum, mouth sprays, mouthwashes, toothpicks, and dental floss.

22. The method of claim 1 wherein the periodontal disease is gingivitis.

23. The method of claim 1 wherein the periodontal disease is periodontitis.

24. A pharmaceutical composition comprising a prophylactically or therapeutically effective amount of a compound of formula I: 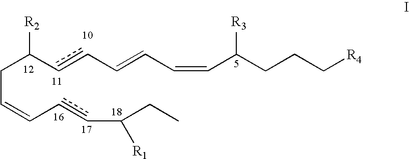
or a pharmaceutically acceptable salt or prodrug thereof;
and a pharmaceutically acceptable carrier,
wherein:
the pharmaceutical composition is in the form of a toothpaste, chewing gum, mouth spray, mouthwash, toothpick, or dental floss;
each 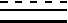
independently designates a double or triple bond;
R¹, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or:
two R on the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O)R, NRC(O)N(R)₂, C(O)OR, C(O)N(R)₂, SO₂R, NRSO₂R, C(O)R, or SO₂N(R)₂.

25. The composition of claim 24 wherein the 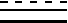
between the C10 and C11 carbons designates a triple bond.

26. The composition of claim 24 wherein the 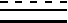
between the C16 and C17 carbons designates a triple bond

27. The composition of claim 24 wherein the 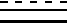
between the C10 and C11 carbons and the C16 and C17 carbons designates a triple bond.

28. The composition of claim 24 wherein each 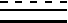
designates a double bond.

29. The composition of claim 24 wherein R¹, R², and R³ are each independently OR, OX¹, SR, SX², N(R)₂, or NHX³.

30. The composition of claim 29 wherein R¹, R², and R³ are each independently OR or OX¹.

31. The composition of claim 24 wherein R is independently selected from hydrogen or an optionally substituted C₁₋₆ aliphatic group.

32. The composition of claim 24 wherein R is hydrogen.

33. The composition of claim 24 wherein R⁴ is C(O)OR, C(O)N(R)₂, or SO₂R.

34. The composition of claim 33 wherein R⁴ is C(O)OR.

35. The composition of claim 24 wherein the C18 carbon has an R configuration.

36. The composition of claim 24 wherein the C5 carbon has an S configuration, the C12 carbon has an R configuration and the C18 carbon has an R configuration.

37. The composition of claim 24 wherein each 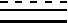
designates a double bond; R¹, R², and R³ are each OH; and R⁴ is C(O)OH.

38. The composition of claim 24 further comprising an antimicrobial compound or a non-steroidal antiinflammatory compound.

39. The composition of claim 24 further comprising a COX-2 inhibitor.

40. The composition of claim 39 wherein the COX-2 inhibitor is selected from the group consisting of celecoxib, rofecoxib, and valdecoxib.
